SEQUENCE LISTING (20 sequences)

SEQ ID NO: 1 - DNA, 21 bp, Artificial Sequence
  Description of Artificial Sequence Synthetic primer
aattctccgaacgtgtcacgt

SEQ ID NO: 2 - DNA, 21 bp, Artificial Sequence
  Description of Artificial Sequence Synthetic primer
aagggcggctttgccaagtgc

SEQ ID NO: 3 - DNA, 21 bp, Artificial Sequence
  Description of Artificial Sequence Synthetic primer
nngtgaagtcaacatgcctgc

SEQ ID NO: 4 - DNA, 21 bp, Artificial Sequence
  Description of Artificial Sequence Synthetic primer
nncctcaccaatgggtccttt

SEQ ID NO: 5 - RNA, 71 nt, Artificial Sequence
  Description of Artificial Sequence Synthetic primer
gggaggacgaugcggaucagccauguuuacgucacuccuugucaauccucaucggcagacgacucgcccga

SEQ ID NO: 6 - RNA, 92 nt, Artificial Sequence
  Description of Artificial Sequence Synthetic primer
gggaggacgaugcggaucagccauguuuacgucacuccuugucaauccucaucggcagacgacucgcccgaaauucuccgaacgugucacgu

SEQ ID NO: 7 - DNA, 21 bp, Artificial Sequence
  Description of Artificial Sequence Synthetic primer
acgugacacguucggagaatt

SEQ ID NO: 8 - RNA, 92 nt, Artificial Sequence
  Description of Artificial Sequence Synthetic primer
gggaggacgaugcggaucagccauguuuacgucacuccuugucaauccucaucggcagacgacucgcccgaaagggcggcuuugccaagugc

SEQ ID NO: 9 - DNA, 21 bp, Artificial Sequence
  Description of Artificial Sequence Synthetic primer
gcacuuggcaaagccgccctt

SEQ ID NO: 10 - RNA, 92 nt, Artificial Sequence
  Description of Artificial Sequence Synthetic primer
gggaggacgaugcggaucagccauguuuacgucacuccuugucaauccucaucggcagacgacucgcccgaaagugaagucaacaugccugc

SEQ ID NO: 11 - RNA, 21 nt, Artificial Sequence
  Description of Artificial Sequence Synthetic primer
gcaggcauguugacuucacuu

SEQ ID NO: 12 - RNA, 92 nt, Artificial Sequence
  Description of Artificial Sequence Synthetic primer
gggaggacgaugcggaucagccauccuuacgucacuccuugucaauccucaucggcagacgacucgcccgaaagggcggcuuugccaagugc

SEQ ID NO: 13 - DNA, 21 bp, Artificial Sequence
  Description of Artificial Sequence Synthetic primer
gcacuuggcaaagccgccctt

SEQ ID NO: 14 - DNA, 32 bp, Artificial Sequence
  Description of Artificial Sequence Synthetic primer
taatacgactcactatagggaggacgatgcgg

SEQ ID NO: 15 - DNA, 16 bp, Artificial Sequence
  Description of Artificial Sequence Synthetic primer
tcgggcgagtcgtctg

SEQ ID NO: 16 - DNA, 71 bp, Artificial Sequence
  Description of Artificial Sequence Synthetic primer
gggaggacgatgcggatcagccatgtttacgtcactccttgtcaatcctcatcggcagacgactcgcccga

SEQ ID NO: 17 - DNA, 37 bp, Artificial Sequence
  Description of Artificial Sequence Synthetic primer
acgtgacacgttcggagaatttcgggcgagtcgtctg

SEQ ID NO: 18 - DNA, 37 bp, Artificial Sequence
  Description of Artificial Sequence Synthetic primer
gcacttggcaaagccgccctttcgggcgagtcgtctg

SEQ ID NO: 19 - DNA, 37 bp, Artificial Sequence
  Description of Artificial Sequence Synthetic primer
gcaggcatgttgacttcactttcgggcgagtcgtctg

SEQ ID NO: 20 - DNA, 31 bp, Artificial Sequence
  Description of Artificial Sequence Synthetic primer
aggacgatgcggatcagccatccttacgtca

(19) United States
(12) Patent Application Publication
(10) Pub. No.: US 2010/0324113 A1
(43) Pub. Date: Dec. 23, 2010

(54) Delivery Method

(76) Inventors: Bruce A. Sullenger, Durham, NC (US); Paloma H. Giangrande, Iowa City, IA (US); James McNamara, Iowa City, IA (US)

Correspondence Address: NIXON & VANDERHYE, PC, 901 NORTH GLEBE ROAD, 11TH FLOOR, ARLINGTON, VA, 22203, US

(21) Appl. No.: 12/227,871

(22) PCT Filed: Jun. 1, 2007
(86) PCT No.: PCT/US07/12927

§ 371 Date: Oct. 7, 2009

Related U.S. Application Data
(60) Provisional application No. 60/809,842, filed on Jun. 1, 2006.

(51) Int. Cl. A61K 31/7052; C07H 21/04; C12N 5/00; A61P 35/00

(52) U.S. Cl. 514/44 A; 536/24.5; 435/375

(57) ABSTRACT

The present invention relates, in general, to siRNA and, in particular, to a method of effecting targeted delivery of siRNAs and to compounds suitable for use in such a method.

This application claims priority from U.S. Prov. Appln. No. 60/809,842, filed Jun. 1, 2006, the entire content of which is incorporated herein by reference.

This invention was made with Government support under Grant No. R01 HL079051 awarded by the National Institutes of Health. The Government has certain rights in the invention.

### TECHNICAL FIELD

The present invention relates, in general, to interfering RNA (RNAi) (e.g., siRNA) and, in particular, to a method of effecting targeted delivery of RNAi's and to compounds suitable for use in such a method.

### BACKGROUND

RNA interference (RNAi) is a cellular mechanism, first described in C. elegans by Fire et al. in 1998, by which 21-23 nt RNA duplexes trigger the degradation of cognate mRNAs (Fire et al, Nature 391(6669):806-811 (1998)). The promise of therapeutic applications of RNAi has been apparent since the first demonstration that exogenous, short interfering RNAs (siRNAs) can silence gene expression via this pathway in mammalian cells (Elbashir et al, Nature 411(6836):494-8 (2001)). The properties of RNAi that are attractive for therapeutics include 1) stringent target gene specificity, 2) relatively low immunogenicity of siRNAs, and 3) simplicity of design and testing of siRNAs.

A critical technical hurdle for RNAi-based clinical applications is the delivery of siRNAs across the plasma membrane of cells in vivo. A number of solutions for this problem have been described including cationic lipids (Yano et al, Clin Cancer Res. 10(22):7721-6 (2004)), viral vectors (Fountaine et al, Curr Gene Ther. 5(4):399-410 (2005), Devroe and Silver, Expert Opin Biol Ther. 4(3):319-27 (2004), Anderson et al, AIDS Res Hum Retroviruses. 19(8):699-706 (2003)), high-pressure injection (Lewis and Wolff, Methods Enzymol. 392:336-50 (2005)), and modifications of the siRNAs (e.g. chemical, lipid, steroid, protein) (Schiffelers et al. Nucleic Acids Res. 32(19):e149 (2004), Urban-Klein et al, Gene Ther. 12(5):461-6 (2005), Soutschek et al, Nature 432(7014):173-8 (2004), Lorenz et al, Bioorg Med Chem Lett. 14(19):4975-7 (2004), Minakuchi et al, Nucleic Acids Res. 32(13):e109 (2004), Takeshita et al, Proc Natl Acad Sci USA. 102(34):12177-82 (2005)). However, most of the approaches described to date have the disadvantage of delivering siRNAs to cells non-specifically, without regard to the cell type.

For in vivo use, it is important to target therapeutic siRNA reagents to particular cell types (e.g., cancer cells), thereby limiting side-effects that result from non-specific delivery as well as reducing the quantity of siRNA necessary for treatment, an important cost consideration. One recent study described a promising method for targeted delivery of siRNAs in which antibodies that bind cell-type specific cell surface receptors were fused to protamine and used to deliver siRNAs to cells via endocytosis (Song et al, Nat. Biotechnol. 23(6):709-17 (2005)).

The present invention relates to a much simpler approach for specific delivery of siRNAs and one that, at least in one embodiment, only uses properties of RNA. With SELEX (systematic evolution of ligands by exponential enrichment), it has been demonstrated that structural RNAs capable of binding a variety of proteins with high affinity and specificity can be identified. The delivery method of the instant invention exploits the structural potential of nucleic acids (e.g., RNA) to target siRNAs to a particular cell-surface receptor and thus to a specific cell type. The invention thus provides a method to specifically deliver nucleic acids that comprise both a targeting moiety (e.g., an aptamer) and an RNA-silencing moiety (e.g., an siRNA) that is recognized and processed by Dicer in a manner similar to the processing of microRNAs.

### SUMMARY OF THE INVENTION

The present invention relates generally to interfering RNA (RNAi) and to a method of delivering same. More specifically, the invention relates to a method of effecting targeted delivery of siRNA that involves the use of a nucleic acid that comprises the siRNA to be delivered and a targeting moiety, wherein the targeting moiety is an aptamer.

Objects and advantages of the present invention will be clear from the description that follows.

### BRIEF DESCRIPTION OF THE DRAWINGS

FIGS. 1A-1D. Schematic and proposed mechanism of action of aptamer-siRNA chimeras. (FIG. 1A) The aptamer-siRNA chimera binds to the cell-surface receptor (light green rectangle), is endocytosed, and subsequently released from the endosome to enter the RNAi pathway. The intracellular silencing pathway is shown for comparison. A pre-microRNA (pre-miRNA) exits the nucleus upon cleavage by Drosha, is recognized by the endonuclease Dicer, which processes the pre-miRNA into a 21 nt mature miRNA. The mature miRNA is subsequently incorporated into the silencing complex (RISC) where it mediates targeted mRNA degradation. (FIG. 1B) Predicted RNA structures for the PSMA-specific aptamer A10 and the A10 aptamer-siRNA chimera derivatives. The region of the A10 aptamer responsible for binding to PSMA is outlined in magenta. This region was mutated in the mutant A10 aptamer, mutA10-Plk1 (mutated bases shown in blue). (The secondary structure of aptamer A10 is shown.) (FIG. 1C) Cell-type specific binding of A10 aptamer-siRNA chimeras. Cell surface binding of fluorescently-labeled aptamer-siRNA chimeras (shown in green) was assessed by Flow cytometric analysis and was found to be restricted to LNCaP cells expressing PSMA. Unstained cells are shown in purple. (FIG. 1D) Cell surface binding of aptamer-siRNA chimeras requires the intact region of A10 responsible for binding to PSMA surface receptor.

FIGS. 2A-2C. A10 aptamer-siRNA chimeras bind specifically to the cell surface antigen, PSMA. (FIG. 2A) Binding of fluorescently-labeled A10 aptamer-siRNA chimeras can be actively competed with excess A10 aptamer. Binding is displayed as % Counts in G1. (FIG. 2B) Cell surface binding of A10 aptamer and to the A10 aptamer-siRNA chimeras to LNCaP cells is disrupted with an antibody specific to human PSMA. Cell surface binding of fluorescently-labeled A10 aptamer and A10 aptamer-siRNA chimeras was assessed by Flow cytometric analysis and is presented as Mean Fluorescence Intensity (MFI). MFI values + or − competitor were used to calculate % Competition. (FIG. 2C) Cell surface binding of A10 aptamer and A10 aptamer-siRNA chimeras to LNCaP cells is reduced upon 5-α-dihydrotestosterone (2 nM DHT) treatment, as a result of reduced PSMA cell surface expression. Binding is displayed as % Counts in G1 (gate 1).

FIGS. 3A-3C. Cell-type specific silencing of genes with aptamer-siRNA chimeras. (FIG. 3A). A10-Plk1 aptamer-siRNA chimera silences Plk1 expression in LNCaP but not PC-3 cells (top panels). Silencing correlates with efficient labeling in LNCaP cells with FITC-labeled A10-Plk1 as determined by Flow cytometric analysis (bottom panels). (FIG. 3B) A10-Bcl-2 aptamer-siRNA chimera silences Bcl-2 expression in LNCaP but not PC-3 cells (top panels). Silencing correlates with labeling of LNCaP cells with FITC-labeled A10-Bcl-2 (bottom panels). (FIG. 3C) A10-Plk1 mediated silencing of Plk1 is reduced upon 5-α-dihydrotestosterone (2 nM DHT) treatment of LNCaP cells.

FIGS. 4A-4C. Aptamer-siRNA chimera-mediated silencing of Plk1 and Bcl-2 genes results in cell-type specific effects on proliferation and apoptosis. (FIG. 4A) Proliferation of PC-3 and LNCaP cells transfected (+ cationic lipids) with either a Plk1 or a control siRNA, or treated (− cationic lipids) with A10 aptamer, or A10 aptamer-siRNA chimeras (A10-CON and A10-Plk1) was determined by incorporation of ³H-thymidine. (FIG. 4B) Apoptosis of PC-3 and LNCaP cells treated with Cisplatin, A10 aptamer, or A10 aptamer-siRNA chimeras (A10-CON and A10-Plk1), or transfected with either a Plk1 or a control siRNA was assessed by Flow cytometric analysis using a PE-conjugated antibody specific for active caspase 3. (FIG. 4C) Apoptosis of PC-3 and LNCaP cells treated with Cisplatin, A10 aptamer, or A10 aptamer-siRNA chimeras (A10-CON and A10-Bcl2) or transfected with either a Bcl2 or a control siRNA was assessed as described above.

FIGS. 5A-5C. Aptamer-siRNA chimera-mediated gene silencing occurs via the RNAi pathway. (FIG. 5A) LNCaP cells transfected with either siRNAs, A10 aptamer, or A10 aptamer-siRNA chimeras (A10-CON and A10-Plk1) in the presence or absence of an siRNA against Dicer. (FIG. 5B) In vitro Dicer assay, RNAs treated with or without Dicer were resolved on a non-denaturing polyacrylamide gel and stained with ethidium bromide. Single-stranded chimeras, ssA10-Plk1 and ssA10-CON (without antisense siRNA). (FIG. 5C) In vitro Dicer assay. Aptamer-siRNA chimeras annealed to the complementary antisense siRNA strand labeled with ³²P, were incubated with or without Dicer and cleavage products were subsequently resolved on a non-denaturing polyacrylamide gel. The antisense siRNAs were not complementary to and thus did not anneal to A10.

FIGS. 6A and 6B. Antitumor activity of A10-Plk1 aptamer-siRNA chimera in a mouse model of prostate cancer. (FIG. 6A) Chimeric RNAs were administered intratumorally in a mouse model bearing either PSMA negative prostate cancer cells, PC-3 (left panel) or PSMA positive prostate cancer cells, LNCaP (right panel) implanted bilaterally into the hind flanks of nude mice. The mean tumor volumes were analyzed using a One-way ANOVA. ***, P<0.0001; **, P<0.001; *, P<0.01. (n=6-8 tumors). (FIG. 6B) Tumor curves for individual LNCaP cell derived tumors showing regression of tumor growth following A10-Plk1 treatment but not treatment with DPBS, A10-CON, or mutA10-Plk1.

FIGS. 7A and 7B. Cell-type specific expression of PSMA. Expression of PSMA was assessed by (FIG. 7A) Flow cytometric analysis and (FIG. 7B) immunoblotting using antibodies specific to human PSMA. PSMA is expressed on the surface of LNCaP prostate cancer cells, but not, PC-3 prostate cancer cells or HeLa cells, a non-prostate derived cancer cell line.

FIGS. 8A and 8B. Relative affinity measurement of A10 and A10 aptamer-siRNA chimera derivatives. (FIG. 8A) Cell surface binding affinities of the fluorescently-labeled RNAs (A10, A10-CON, and A10-Plk1) were assessed by Flow cytometric analysis. (FIG. 8B) Plat of % MFI (mean fluorescence intensity) in G1 for data in part (FIG. 8A). The, relative affinities of A10 and the aptamer-siRNA chimeras for the LNCaP cell surface, were determined by incubating increasing amounts of fluorescently labeled A10, A10-CON or A10-Plk1 RNAs with LNCaP cells. Cellular fluorescence was measured with flow cytometry. The aptamer-siRNA chimeras and A10 were found to have comparable affinities for the LNCaP cell surface.

FIGS. 9A and 9B. Gene silencing mediated by functional siRNAs against Polo-like kinase 1 (Plk1) and Bcl2. Gene silencing was achieved by cationic lipid delivery of siRNA specific to either (FIG. 9A) human Plk1 or (FIG. 9B) human bcl-2 to PC-3 and LNCaP cells. Silencing was assessed by Flow cytometric analysis (top panels) and immunoblotting (bottom panel).

FIGS. 10A and 10B. siRNA-mediated silencing of Dicer. Silencing of Dicer gene expression was evaluated by (FIG. 10A) flow cytometry and by (FIG. 10B) enzyme-linked immunosorbant assay (ELISA) using an antibody specific for human Dicer. HeLa cells were transfected with a control, non-silencing siRNA, or an siRNA against human Dicer. Silencing by the Dicer siRNA was specific and resulted in >80% reduction in Dicer gene expression.

FIGS. 11A and 11B. Aptamer-siRNA chimeras do not trigger an interferon response. (FIG. 11A) PC-3 and (FIG. 11B) LNCaP cells treated with siRNAs (con, Plk1, or Bcl-2), A10 aptamer, or aptamer-siRNA chimeras (A10-CON, A10-Plk1, or A10-Bcl2) were assessed for production of interferon-β (INF-B) by enzyme-linked immunosorbant assay (ELISA) using an antibody specific for INF-β. Cells treated with the interferon inducer Poly(I:C) were used as a positive control in this experiment.

### DETAILED DESCRIPTION OF THE INVENTION

The present invention relates to a method of effecting targeted delivery of RNAi's (e.g., siRNAs and short hairpin RNAs (shRNA)). This method can be used, for example, to target delivery of siRNAs to specific cell types (e.g., cells bearing a particular protein, carbohydrate or lipid (for example, a certain cell-surface receptor)). In contrast to most delivery methods described to date, the method disclosed herein can be carried out using a compound that comprises only RNA. The molecule used is a chimeric molecule comprising a nucleic acid targeting moiety (e.g., an aptamer) linked to an RNA silencing moiety (e.g., an siRNA (comprising modified or unmodified RNA)). (In accordance with the invention, the targeting moiety (e.g., aptamer) can comprise RNA, DNA or any modified nucleic acid based oligonucleotide.)

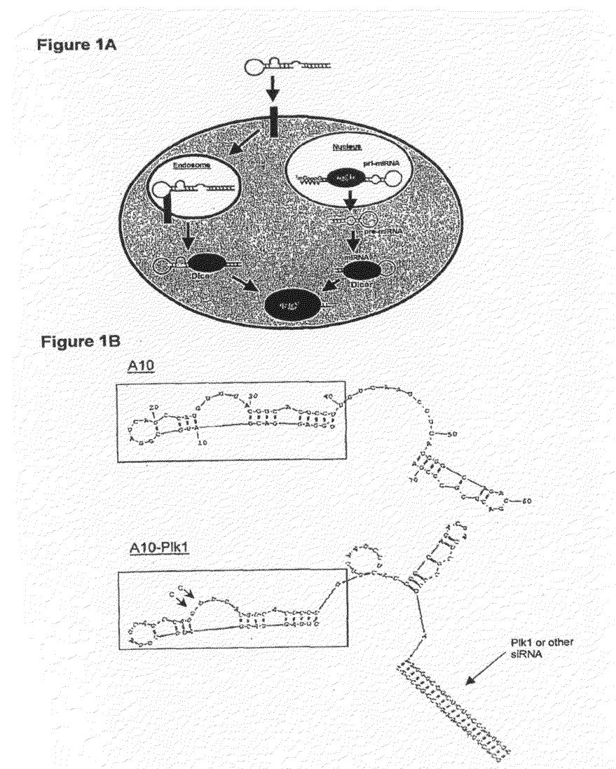

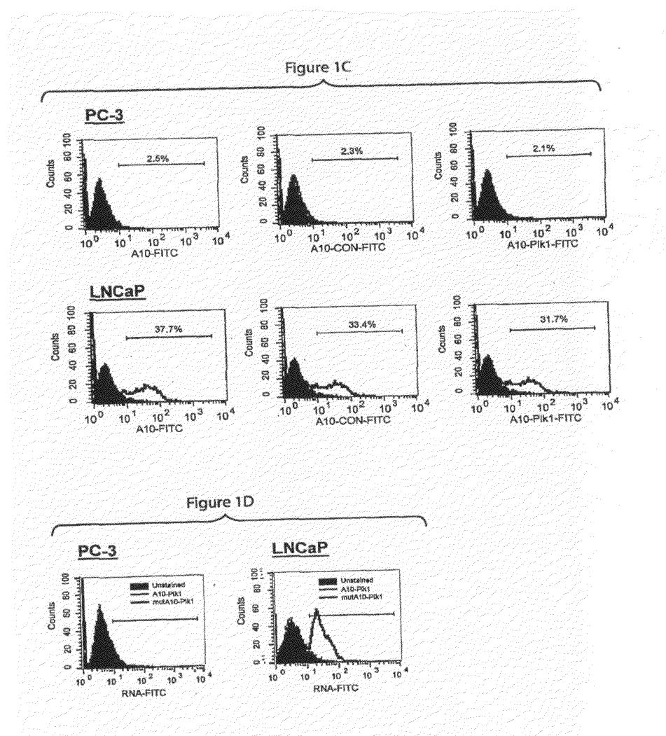

The invention is exemplified below with reference to aptamer-siRNA chimeric RNAs that: i) specifically bind prostate cancer cells (and vascular endothelium of most solid tumors expressing the cell-surface receptor PSMA (due to the use of an RNA aptamer selected against human PSMA (A10) (Lupold et al, Cancer Res. 62(14):4029-33 (2002)), and ii) deliver therapeutic siRNAs that target polo like kinase 1 (Plk1) (Reagan-Shaw and Ahmad, FASEB J. 19(6):611-3 (2005)) and Bcl2 (Yang et al, Clin Cancer Res. 10(22):7721-6 (2004)) (two survival genes overexpressed in most human tumors (Takai et al, Oncogene 24(2):287-291 (2005), Eckerdt et al, Oncogene 24(2):267-76 (2005), Cory and Adans, Cancer Cell 8(1):5-6 (2005)). These chimeric RNAs act as substrates for Dicer, thus directing the siRNAs into the RNAi pathway and silencing their cognate mRNAs (FIG. 1A). (Thus the chimeric aptamer-siRNAs can actually be viewed as aptamer-presiRNAs as siRNAs result from Dicer cleavage.)The particular reagents described in the Example below are expected to have application in treating prostate and other cancers.

The invention, however, is not limited to chimeras specific for PSMA. Rather, the present approach can be adapted to generate therapeutics to treat a wide variety of diseases, in addition to cancer. The two requirements for this approach for a given disease are that silencing specific genes in a defined population of cells produces a therapeutic benefit and that surface receptors are expressed specifically on the cell population of interest that can deliver RNA ligands intracellularly. Many diseases satisfy both of these requirements (examples include CD4+ T-cell's for HIV inhibition, insulin receptor and diabetes, liver receptor cells and hepatitis genes, etc).

Appropriate targeting and silencing moieties can be designed/selected using methods known in the art based on the nature of the molecule to be targeted and gene(s) to be silenced (see Nimjee et al, Annu. Rev. Med. 56:555-83 (2005) and U.S. Publication Appln. 20060105975). The chimeras can be synthesized using RNA synthesis methods known in the art (e.g. via chemical synthesis or via RNA polymerases). Short RNA aptamers (25-35 bases) that bind various targets with high affinities have been described (Pestourie et al, Biochimie (2005), Nimjee et al, Annu. Rev. Med. 56:555-83 (2005)). Chimeras designed with such short aptamers have a long strand of approximately 45-55 bases. Chemically synthesized. RNA is amenable to various modifications, such as pegylation, that can be used to modify its in vivo half-life and bioavailability. (See also, for example, U.S. Application Nos. 20020086356, 20020177570, 20060105975, and 20020055162, and U.S. Pat. Nos. 6,197,944, 6,590,093, 6,399,307, 6,057,134, 5,939,262, and 5,256,555, in addition, see also Manoharan, Biochem. Biophys. Acta 1489:117 (1999); Herdewijn, Antisense Nucleic Acid Drug Development 10:297 (2000); Maier et al, Organic Letters 2:1819 (2000), and references cited therein.)

The chimeras of the invention can be formulated into pharmaceutical compositions that can include, in addition to the chimera, a pharmaceutically acceptable carrier, diluent or excipient. The precise nature of the composition will depend, at least in part, on the nature of the chimera and the route of administration. Optimum dosing regimens can be readily established by one skilled in the art and can vary with the chimera, the patient and the effect sought. Generally, the chimera can be administered IV, IM, IP, SC, or topically, as appropriate.

In practice, the targeted delivery method of the instant invention can avoid adverse side-effects associated with delivery of siRNAs to non-targeted cells. For example, siRNAs are known to activate toll-like receptors within plasmacytoid dendritic cells, leading to interferon secretion, which can result in various adverse symptoms (Sledz et al, Nat. Cell Biol. 5(9):834-9 (2003), Kariko et al, J. Immunol. 172(11):6545-9 (2004)). In the case of delivering siRNAs that trigger apoptosis, another danger that is avoided by use of the present approach is the killing of healthy cells. Treatments involving systemic delivery of chimera of the invention can be expected to require substantially less targeted (as compared with non-targeted) reagent (e.g., siRNA) due to the reduction in uptake by non-targeted cells. Thus, the method described can substantially reduce the cost of the therapy.

As RNA is believed to be less immunogenic than protein, the chimeric RNAs of the invention can be expected to produce less non-specific activation of the immune system than protein-mediated delivery approaches. This may be an important difference as a number of proteins currently used for therapeutics are known to occasionally cause dangerous allergic reactions especially following repeated administration (Park, Int. Anesthesiol. Cl9in. 42(3):135-45 (2004), Shepherd, Mt. Sinai J. Med. 70(2):113-25 (2003)).

Kim et al., have proposed that Dicer-mediated processing of RNAs may result in more efficient incorporation of resulting siRNAs into RISC complexes (Kim et al, Nat. Biotechnol. 23(2):222-6 (2005)). This suggestion is based on the observation that longer double-stranded RNAs (˜29 bps), which are processed by Dicer, deplete their cognate mRNAs at lower concentrations than siRNAs (19-21 bps), which are not processed by Dicer. Thus, while not wishing to be bound by theory, it is speculated that because chimeras of the invention are processed by Dicer, they may be more potent in terms of gene-silencing ability than dsRNA of 19-21 bps that are not processed.

Advantageously chimeras of the invention:

i) recognize a cell surface receptor,

ii) internalize into a cell expressing the receptor, and

iii) are recognized by miRNA or siRNA processing machinery (such as Dicer). Further, the cleavage siRNA product can be loaded into an RNAi or miRNA silencing complex (such as RISC). Thus, at least in a preferred embodiment, the processing of chimeras of the invention mimic how cells recognize and process miRNAs (e.g., the instant chimeric RNAs can be substrates for Dicer). (See also McNamara et al, Nature Biotechnology 24:1005-1015 (2006).)

Certain aspects of the invention can be described in greater detail in the non-limiting Example that follows.

### EXAMPLE

### Experimental Details

Unless otherwise noted, all chemicals were purchased from Sigma-Aldrich Co., all restriction enzymes were obtained from New England BioLabs, Inc. (NEB), and all cell culture products were purchased from Gibco BRL/Life Technologies, a division of Invitrogen Corp.

| | |
|---|---|
| siRNAs | |
| con siRNA target sequence: | AATTCTCCGAACGTGTCACGT |
| Plk1 siRNA target sequence: | AAGGGCGGCTTTGCCAAGTGC |
| Bcl-2 siRNA target sequence: | NNGTGAAGTCAACATGCCTGC |
| Dicer siRNA target sequence | NNCCTCACCAATGGGTCCTTT |

(where“N” is any of A, T, G or C)

Fluorescent siRNAs labeled with FITC at the 5′ end of the antisense strand were purchased from Dharmacon.

Aptamer-siRNA Chimeras
A 10:
5′GGGAGGACGAUGCGGAUCAGCCAUGUUUACGUCACUCCUUGUCAAUCC
UCAUCGGCAGACGACUCGCCCGA3′
A 10-CON Sense Strand:
5′GGGAGGACGAUGCGGAUCAGCCAUGUUUACGUCACUCCUUGUCAAUCC
UCAUCGGCAGACGACUCGCCCGAAAUUCUCCGAACGUGUCACGU3′
A10-CON Antisense siRNA:
5′ACGUGACACGUUCGGAGAAdTdT3′
A10-Plk1 Sense Strand:
5′GGGAGGACGAUGCGGAUCAGCCAUGUUUACGUCACUCCUUGUCAAUCC
UCAUCGGCAGACGACUCGCCCGAAAGGGCGGCUUUGCCAAGUGC3′
A10-Plk1 Antisense siRNA:
5′GCACUUGGCAAAGCCGCCCdTdT3′
A10-Bcl-2 Sense Strand:
5′GGGAGGACGAUGCGGAUCAGCCAUGUUUACGUCACUCCUUGUCAAUCC
UCAUCGGCAGACGACUCGCCCGAAAGUGAAGUCAACAUGCCUGC3′
A10-Bcl-2 Antisense siRNA:
5′GCAGGCAUGUUGACUUCACUU-3′
mutA10-Plk1 Sense Strand:
5′GGGAGGACGAUGCGGAUCAGCCAUCCUUACGUCACUCCUUGUCAAUCC
UCAUCGGCAGACGACUCGCCCGAAAGGGCGGCUUUGCCAAGUGC3′
A10-Plk1 Antisense siRNA:
5′GCACUUGGCAAAGCCGCCCdTdT3′
A10 5′-primer:
5′TAATACGACTCACTATAGGGAGGACGATGCGG3
A10 3′-primer:
5′TCGGGCGAGTCGTCTG3′
A10 template primer:
5′GGGAGGACGATGCGGATCAGCCATGTTTACGTCACTCCTTGTCAATCC
TCATCGGCAGACGACTCGCCCGA3′
Control siRNA 3′-primer:
5′ACGTGACACGTTCGGAGAATTTCGGGCGAGTCGTCTG3′
Plk1 siRNA 3′-primer:
5′GCACTTGGCAAAGCCGCCCTTTCGGGCGAGTCGTCTG3′
Bcl-2 siRNA 3′-primer:
5′GCAGGCATGTTGACTTCACTTTCGGGCGAGTCGTCTG3′
A10 mutant primer:
5′AGGACGATGCGGATCAGCCATCCTTACGTCA3′

Double-stranded DNA templates were generated by PCR as follows. The A10 template primer was used as a template for the PCRs with the A10 5′-primer and one of the following 3′-primers: A10 3′-primer (for the A10 aptamer), Control siRNA 3′-primer (for the A10-CON chimera), Plk1 siRNA 3′-primer (for the A10-Plk1 chimera) or Bcl-2 siRNA 3′-primer (for the A10-Bcl-2 chimera). Templates for transcription were generated in this way or by cloning these PCR products into a T-A cloning vector (pGem-t-easy, Promega (Madison, Wis.)) and using the clones as templates for PCR with the appropriate primers.

The DNA encoding the mutA10-Plk1 chimera was prepared by sequential PCRs. In the first reaction, the A10 template primer was used as the template with the A10 mutant primer as the 5′-primer and the Plk1 siRNA 3′-primer as the 3′-primer. The product of this reaction was purified and used as the template for a second reaction with the A10 5′-primer and the Plk 1 siRNA 3′-primer. The resulting PCR product was cloned into pGem-t-easy and sequenced. This clone was used as the template in a PCR with the A10 5′-primer and the Plk-1 3′-primer to generate the template for transcription. Fluorescent aptamer and aptamer-siRNA chimeras were in vitro transcribed in the presence of 5′-(FAM)(spacer 9)-G-3′ (FAM-labeled G) (TriLink) as described below.

### In Vitro Transcriptions

Transcriptions were set up either with or without 4 mM FAM-labeled G. For a 250 μL transcription reactions: 50 μL 5× T7 RNAP Buffer optimized for 2′F transcriptions (20% w/v PEG 8000, 200 mM Tris-HCl pH 8.0, 60 mM MgCl₂, 5 mM spermidine HCl, 0.01% w/v triton X-100, 25 mM DTT), 25 μL 10× 2′F-dNTPs (30 mM, 2′F-CTP, 30 mM 2′F-UTP, 10 mM 2′OH-ATP, 10 mM 2′OH-GTP), 2 μL IPPI (Roche), 300 pmoles aptamer-siRNA chimera PCR template, 3 μL T7(Y639F) polymerase (Padilla and Sousa, Nucleic Acids Res. 27(6):1561-3 (1999)), bring up to 250 μL with milliQ H₂O.

### Predicting RNA Secondary Structure

RNA Structure Program version 4.1 (rna.chem.rochester.edu/RNAstructure) was used to predict the secondary structures of A10 aptamer, A10-3, and A10 aptamer-siRNA chimera derivatives. The most stable structures with the lowest free energies for each RNA oligo were compared.

### Cell Culture

HeLa cells were maintained at 37° C. and 5% CO₂ in DMEM supplemented with 10% fetal bovine serum. Prostate carcinoma cell lines LNCaP (ATCC # CRL-1740) and PC-3 (ATCC # CRL-1435) were grown in RPMI 1640 and Ham's F12-K medium respectively, supplemented with 10% fetal bovine serum (FBS).

### PSMA Cell-Surface Expression

PSMA cell-surface expression was determined by Flow cytometry and/or immunoblotting using antibodies specific to human PSMA. Flow cytometry: HeLa, PC-3, and LNCaP cells were trypsinized, washed three times in phosphate buffered saline (PBS), and counted using a hemocytometer. 200,000 cells (1×10⁶ cells/mL) were resuspended in 500 μl of PBS+4% fetal bovine serum (FBS) and incubated at room temperature (RT) for 20 min. Cells were then pelleted and resuspended in 100 μL of PBS+4% FBS containing 20 μg/mL of primary antibody against PSMA (anti-PSMA 3C6: Northwest Biotherapeutics) or 20 μg/mL of isotype-specific control antibody. After a 40 min incubation at RT cells were washed three times with 500 μL of PBS+4% FBS and incubated with a 1:500 dilution of secondary antibody (anti-mouse IgG-APC) in PBS+4% FBS for 30 min at RT. Cells were washed as detailed above, fixed with 400 μL of PBS+1% formaldehyde, and analyzed by Flow cytometry. Immunoblotting: HeLa, PC-3, and LNCaP cells were collected as described above. Cell pellets were resuspended in 1× RIPA buffer (150 mM NaCl, 50 mM Tris-HCl pH 8.0, 1 mM EDTA, 1% NP-40) containing 1× protease and phosphatase inhibitor cocktails (Sigma) and incubated on ice for 20 min. Cells were then pelleted and 25 μg of total protein from the supernatants were resolved on a 7.5% SDS-PAGE gel. PSMA was detected using an antibody specific to human PSMA (anti-PSMA 1D11; Northwest Biotherapeutics).

### Cell-Surface Binding of Aptamer-siRNA Chimeras

PC-3 or LNCaP cells were trypsinized, washed twice with 500 μL PBS, and fixed in 400 μL of FIX solution (PBS+1% formaldehyde) for 20 min at RT. After washing cells to remove any residual trace of formaldehyde, cell pellets were resuspended in 1× Binding Buffer (1×BB) (20 mM HEPES pH 7.4, 150 mM NaCl, 2 mM CaCl₂, 0.01% BSA) and incubated at 37° C. for 20 min. Cells were then pelleted and resuspended in 50 μL of 1×BB (pre-warmed at 37° C.) containing either 400 nM FAM-G labeled A10 aptamer or 400 nM FAM-G labeled aptamer-siRNA chimeras. Due to the low incorporation efficiency of FAM-G during the transcription reaction, for comparison of A10-Plk1 and mutA10-Plk1 cell surface binding up to 10 μM of FAM-G labeled aptamer chimeras were used. Concentrations of FAM-G labeled aptamer and aptamer-siRNA chimeras for the relative affinity measurements varied from 0 to 4 μM. Cells were incubated with the RNA for 40 min at 37° C., washed three times with 500 μL of 1×BB pre-warmed at 37° C., and finally resuspended in 400 μL of FIX solution pre-warmed at 37° C. Cells were then assayed using Flow cytometry as detailed above and the relative cell surface binding affinities of the A10 aptamer and A10 aptamer-siRNA chimera derivatives were determined.

### Cell-Surface Binding Competition Assays

LNCaP cells were prepared as detailed above for the cell-surface binding experiments. 4 μM of FAM-G labeled A10 aptamer or A10 aptamer-siRNA chimera derivatives were competed with either unlabelled A10 aptamer (concentration varied from 0 to 4 μM) in 1×BB pre-warmed at 37° C. or 2 μg of anti-PSMA 3C6 antibody in PBS+4% FBS. Cells were washed three times as detailed above, fixed in 400 μL of FIX (PBS+1% formaldehyde), and analyzed by Flow cytometry.

### 5-α-Dihydrotestosterone (DHT) Treatment

LNCaP cells were grown in RPMI 1640 medium containing 5% charcoal stripped serum for 24 h prior to addition of 2 nM 5-α-dihydrotestosterone (DHT) (Sigma) in RPMI 1640 medium containing 5% charcoal stripped FBS for 48 h. PSMA expression was assessed by immunoblotting as detailed above. PSMA cell surface expression was analyzed by flow cytometry as detailed above. Cell-surface binding of FAM-G labeled A10 aptamer and FAM-G labeled A10-CON, A10-Plk1, and mutA10-Plk1 aptamer chimeras was done as detailed above using 40 μM of FAM-G labeled RNA.

### Gene Silencing Assay

siRNA: (Day 1) PC-3 and LNCaP cells were seeded in 6-well plates at 60% confluency. Cells were transfected with either 200 nM or 400 nM siRNA on day 2 and 4 using Superfect Reagent (Qiagen) following manufacturer's recommendations. Cells were collected on day 5 for analysis. A10 aptamer and A10 aptamer-siRNA chimeras: (Day 1) PC-3 and LNCaP cells were seeded in 6-well plates at 60% confluency. Cells were treated with 400 nM A10 aptamer or A10 aptamer-siRNA chimeras on day 2 and 4. Cells were collected on day 5 for analysis.

Gene silencing was assessed by flow cytometry or immunoblotting using antibodies specific to human Plk1 (Zymed) and human Bcl-2 (Zymed) respectively. Flow cytometry: PC-3 and LNCaP cells were trypsinized, washed three times in phosphate buffered saline (PBS), and counted using a hemocytometer. 200,000 cells (5×10⁵ cells/mL) were resuspended in 400 μl of PERM/FIX buffer (Pharmingen) and incubated at room temperature (RT) for 20 min. Cells were then pelleted and washed three times with 1× PERM/WASH buffer (Pharmingen). Cells were then resuspended in 50 μL 1× PERM/WASH buffer containing 20 μg/mL of primary antibody against either human Plk1, or human Bcl-2, or 20 μg/mL of isotype-specific control antibody. After 40 min incubation at RT, cells were washed three times with 500 μL 1× PERM/WASH buffer and incubated with a 1:500 dilution of secondary antibody (anti-mouse IgG-APC) in 1× PERM/WASH for 30 min at RT. Cells were washed as detailed above and analyzed by Flow cytometry. Immunoblotting: LNCaP cells were transfected with control siRNA, or siRNAs to either Plk1 or Bcl-2 as described above. Cells were trypsinized, washed in PBS, and cell pellets were resuspended in 1× RIPA buffer and incubated on ice for 20 min. Cells were then pelleted and 50 μg of total protein from the supernatants were resolved on either 8.5% SDS-PAGE gel for Plk1 or a 15% SDS-PAGE gel for Bcl-2. Plk1 was detected using an antibody specific to human Plk1 (Zymed). Bcl-2 was detected using an antibody specific to human Bcl-2 (Dykocytomation).

### Proliferation (DNA Synthesis) Assay

PC-3 and LNCaP cells previously treated with siRNAs or aptamer-siRNA chimeras as detailed above, were trypsinized and seeded in 12-well plates at ˜20,000 cells/well. Cells were then forced into a G1/S block by addition of 0.5 μM hydroxy urea (HU). After 21 hr cells were released from the HU block by addition of media lacking HU and incubated with media containing ³H-thymidine (1 μCi/mL medium) to monitor DNA synthesis. After 24 hr incubation in the presence of media containing ³H-thymidine, cells were washed twice with PBS, washed once with 5% w/v trichloroacetic acid (TCA) (VWR), collected in 0.5 mL of 0.5N NaOH (VWR) and placed in scintillation vials for measurement of ³H-thymidine incorporation.

### Active Caspase 3 Assay

PC-3 or LNCaP cells were either transfected with siRNAs to Plk1 or Bcl-2 or treated with A10 aptamer-siRNA chimeras as described above. Cells were also treated with medium containing 100 μM (1×) or 200 μM (2×) cisplatin for 30 hr as a positive control for apoptosis. Cells were then fixed and stained for active caspase 3 using a PE-conjugated antibody specific to cleaved caspase 3 as specified in manufacturer's protocol (Pharmingen). Flow cytometric analysis was used to quantitate % PE positive cells as a measure of apoptosis.

Dicer siRNA

HeLa cells were seeded in 6-well plates at 200,000 cells per well. After 24 hr, cells were transfected with either 400 nM of control siRNA or an siRNA against human dicer using Superfect Reagent as described above. Cells were then collected and processed for Flow cytometric analysis using an antibody specific for human Dicer (IMX-5162; IMGENEX) as described above for analysis of Plk1 and Bcl-2 by Flow.

Enzyme-Linked Immunosorbant Assay (ELISA)

HeLa cells were seeded in 6-well plates at 200,000 cells per well. After 24 hr, cells were transfected with either 400 nM of control, non-silencing siRNA or an siRNA against human dicer using Superfect Reagent as described above. Cells were then collected and lysed in 1× RIPA buffer containing 1× protease and phosphatase inhibitor cocktail (Sigma) for 20 min on ice. 100 μL of cell lysates were then added to each ELISA 96-well plate and incubated at RT for 24 h. Wells were washed three times with 300 μL of 1× RIPA and incubated with 100 μL of 1:200 dilution of primary antibody to human Dicer in 1× RIPA for 2 hr. Wells were washed as above, and incubated with 100 μL of 1:200 dilution of secondary anti-rabbit IgG-HRP antibody in 1× RIPA for 1 hr. Wells were washed as above prior to addition of 100 μL of TMB substrate solution (PBL Biomedical Laboratories). After 20 min 50 μL of 1M H₂SO₄ (Stop Solution) was added to each well and OD₄₅₀-OD₅₄₀ was determined using a plate reader.

### In Vivo Dicer Assay

LNCaP cells were seeded in 6-well plates at 200,000 cells per well. After 24 hr, cells were co-transfected with either 400 nM of control siRNA, 400 nM of Plk1 siRNA, 400 nM A10 aptamer, or 400 nM of A10 aptamer-siRNA chimeras alone or with an siRNA to human Dicer, using Superfect Transfection Reagent as described above. Cells were then collected and processed for Flow cytometric analysis using an antibody specific for human Plk1 as described above.

### In Vitro Dicer Assay

1-2 μg of A10 aptamer or A10 aptamer-siRNA chimeras were digested using recombinant dicer enzyme following manufacturer's recommendations (Recombinant Human Turbo Dicer Kit; GTS) (Myers et al, Nat. Biotechnol. 21(3):324-S (2003)). ssA10-CON and ssA10-Plk1 correspond to the aptamer-siRNA chimeras without the complementary antisense siRNA strand. Digests were then resolved on a 15% non-denaturing PAGE gel and stained with ethidium bromide prior to visualization using the GEL.DOCXR (BioRad) gel camera. Alternatively, 1-2 μg of A10 aptamer or A10 aptamer-siRNA chimera sense strands were annealed to ³²P-end-labeled complementary antisense siRNAs (probe). The siRNAs were end-labeled using T4 polynucleotide kinase (NEB) following manufacturer's recommendations. The antisense siRNA were not complementary to the A10 aptamer. A10 or the annealed chimeras (A10-CON or A10-Plk1) were incubated with or without dicer enzyme and subsequently resolved on a 15% non-denaturing PAGE gel as described above. The gel was dried and exposed to BioMAX MR film (Kodak) for 5 min.

### Interferon Assay

Secreted IFN-β from treated and untreated PC-3 and LNCaP cells was detected using a human Interferon beta ELISA kit following manufacturer's recommendations (PBL Biomedical Laboratories). Briefly, cells were seeded at 200,000 cells/well in 6 well plates. Twenty-four hours later, cells were either transfected with a mixture of Superfect Transfection Reagent (Qiagen) plus varying amounts of Poly(I:C) (2.5, 5, 10, 15 μg/ml) as a positive control for Interferon beta, or with a mixture of Superfect Transfection Reagent and either con siRNAs or siRNAs to Plk1 or Bcl2 (200 nm or 400 nm). In addition, cells were treated with 400 nM each of A10 aptamer and A10 aptamer-siRNA chimeras as described above. 48 hr after the various treatments 100 μL of supernatant from each treatment group was added to a well of a 96-well plate and incubated at RT for 24 hr. Presence of INF-beta in the supernatants was detected using an antibody specific to human INF-beta following manufacturer's recommendations.

### In Vivo Experiments

Athymic nude mice (nu/nu) were obtained from the Cancer Center Isolation Facility (CCIF) at Duke University and maintained in a sterile environment according to guidelines established by the US Department of Agriculture and the American Association for Accreditation of Laboratory Animal Care (AAALAC). This project was approved by the Institutional Animal Care and Utilization Committee (IAUCUC) of Duke University. Athymic mice were inoculated with either 5×10⁶ (in 100 μl of 50% matrigel) in vitro propagated PC-3 or LNCaP cells subcutaneously injected into each flank. Approximately, thirty-two non-necrotic tumors for each tumor type which exceeded 1 cm in diameter were randomly divided into four groups of eight mice per treatment group as follows: group 1, no treatment (DPBS); group 2, treated with A10-CON chimera, (200 pmols/injection×10); group 3, treated with A10-Plk1 chimera (200 pmols/injection×10); group 4, treated with mut-A10-Plk1 chimera (200 pmols/injection×10). Compounds were injected intratumorally in 75 μL volumes every other day for a total of 20 days. Day 0 marks the first day of injection. The small volume injections are small enough to preclude the compounds being forced inside the cells due to a non-specific high-pressure injection. Tumors were measured every three days with calipers in three dimensions. The following formula was used to calculate tumor volume: V_{T}=(WXLXH)×0.5236 (W, the shortest dimension; L, the longest dimension). The growth curves are plotted as the means tumor volume±SEM. The experiment was terminated by euthanasia 3 days after the last treatment when the tumors were excised and formalin fixed for immunohistochemistry.

### Statistical Analysis

Statistical analysis was conducted using a one-way ANOVA. A P-value of 0.05 or less was considered to indicate a significant difference. In addition to a one-way ANOVA, two-tailed unpaired t tests were conducted to compare each treatment group to every other. For tumors expressing PSMA, Group 3 (A10-Plk1) was significantly different from group 1 (DPBS), group 2 (A10-CON), and group 4 (mutA10-Plk1), P<0.01, on Days 12, 15, 18, and 21. Group 2 (A10-CON) and group 4 (mutA10-Plk1) were not significantly different from the DPBS control group, P>0.05, at any point during the treatment. For PSMA negative tumors, there was no significant difference between the groups.

### Results

### A10 Aptamer-siRNA Chimeras.

Aptamer-siRNA chimeric RNAs were generated in order to specifically target siRNAs to cells expressing the cell-surface receptor PSMA. The aptamer portion of the chimera (A10) mediates binding to PSMA. The siRNA portion targets the expression of survival genes such as Plk1 (A10-Plk1) and Bcl2 (A10-Bcl2). A non-silencing siRNA was used as a control (A10-CON). The RNA Structure Program (version 4.1) was used to predict the secondary structures of A10 and the A10 aptamer-siRNA chimera derivatives (FIG. 1B). To predict the region of A10 responsible for binding to PSMA, a comparison was made of the predicted secondary structure for A10 to that of a truncated A10 aptamer, A10-3 (data not shown) (Lupold et al, Cancer Res. 62(14):4029-33 (2002)). Because A10-3 also binds PSMA, the structural component retained in A10-3 is likely to be that necessary for binding PSMA (boxed in magenta in FIG. 1B). The predicted structures of the aptamer-siRNAs retain this predicted PSMA-binding component, suggesting that they also retain PSMA-binding (FIG. 1B, shown for A10-Plk1). As a control, two point mutations were made within this region (mutA10-Plk1), which are predicted to disrupt the secondary structure of the putative PSMA-binding portion of the A10 aptamer (FIG. 1B, shown in blue).

### A10 Aptamer-siRNA Chimeras Bind Specifically to PSMA Expressing Cells.

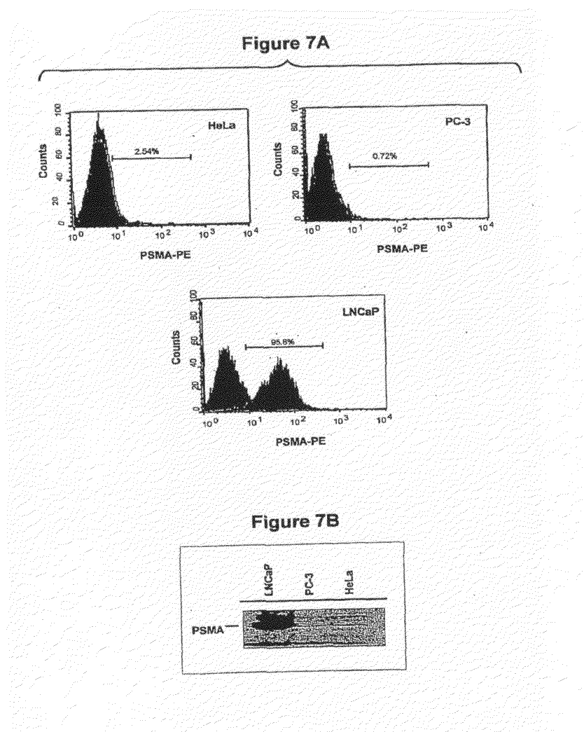

First, the ability of the A10 aptamer-siRNA chimeras to bind the surface of cells expressing PSMA was tested. Previously, PSMA has been shown to be expressed on the surface of LNCaP cells, but not the surface of PC-3 cells (a distinct prostate cancer cell), a finding that was verified with flow cytometry and immunoblotting (FIG. 7). To determine whether the A10 aptamer-siRNA chimeras can bind the surface of cells expressing PSMA, fluorescently labeled

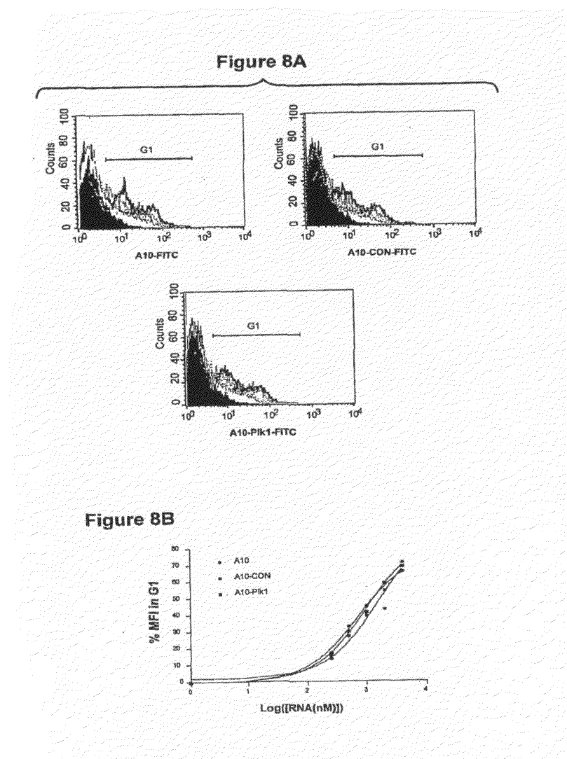

A10, A10-CON, or A10-Plk1 were incubated with either LNCaP or PC-3 cells (FIG. 1C). Binding of A10 and A10 aptamer-siRNA chimeras was specific to LNCaP cells and was dependent on the region of A10 aptamer predicted to bind PSMA as the mutA10-Plk1 was unable to bind (FIG. 1D). Furthermore, the aptamer-siRNA chimeras and the A10 aptamer were found to bind to the surface of LNCaP cells with comparable affinities (FIG. 8).

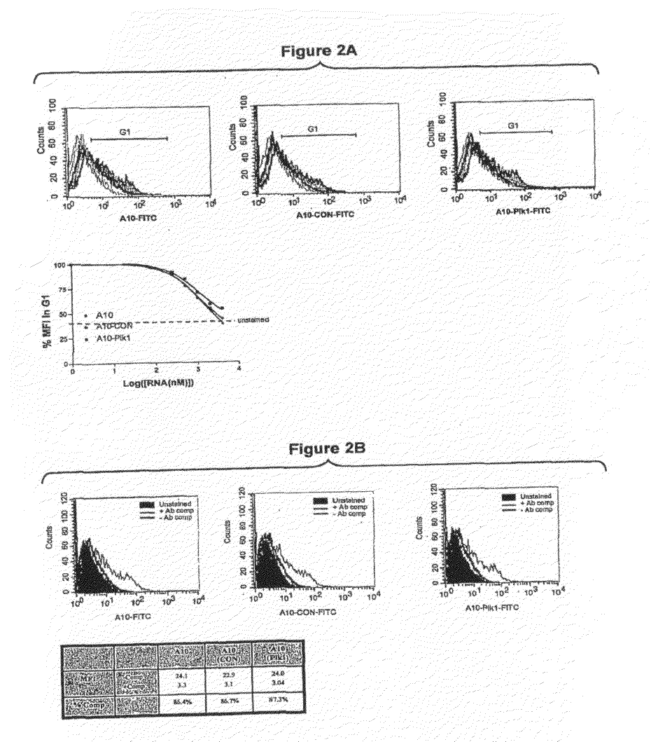

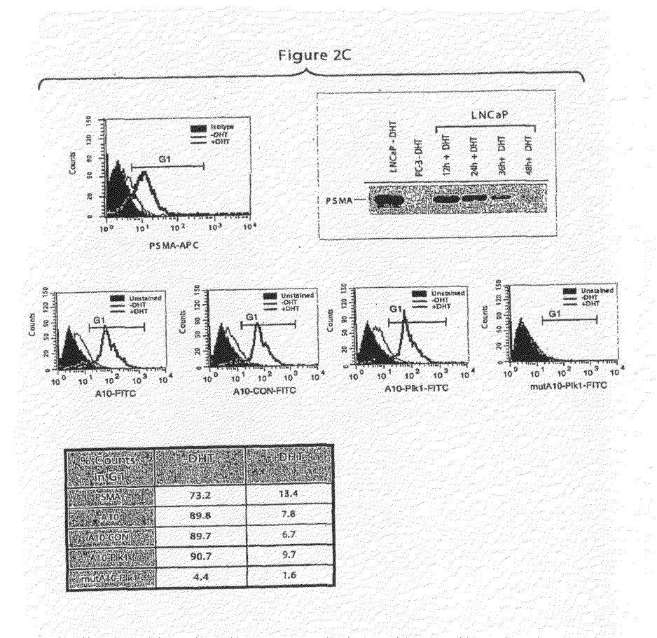

To verify that the A10 aptamer-siRNA chimeras were indeed binding to PSMA, LNCaP cells were incubated with (1 μM) of either fluorescently labeled A10, A10-CON, or A10-Plk1 RNA and competed with increasing amounts (from 0 μM to 4 μM) of unlabeled A10 aptamer (FIG. 2A) or with an antibody specific for human PSMA (FIG. 2B). Bound fluorescently labeled RNAs in the presence of increasing amounts of competitor were assessed using flow cytometry. Binding of the labeled A10 aptamer and A10 aptamer-siRNA chimeras (A10-CON and A10-Plk1) to LNCaP cells was equally competed with either unlabeled A10 or the anti-PSMA antibody indicating that these RNAs are binding PSMA on the surface of LNCaP cells. To further confirm that the target of the aptamer-siRNA chimeras is indeed PSMA, binding of the chimeras was tested to LNCaP cells pre-treated with 5-α-dihydrotestosterone (DHT) since DHT has been shown to reduce the expression of PSMA (Israeli et al, Cancer Res. 54(7):1807-11 (1994)). DHT-mediated inhibition of PSMA gene expression was assessed by flow cytometry and immunoblotting (FIG. 2C, top panels). Treatment of LNCaP cells with 2 nM DHT for 48 h greatly reduced the expression of PSMA. Cell surface expression of PSMA was reduced from 73.2% to 13.4% as determined by flow cytometry and correlated with reduced binding of A10 and A10 aptamer-siRNA chimeras (A10-CON and A10-Plk1) to LNCaP cells (FIG. 2C). As expected, mutA10-Plk1 did not bind to the surface of LNCaP cells either in the presence of absence of DHT treatment (FIG. 2C).

### Aptamer-siRNA Chimeras Specifically Silence Gene Expression.

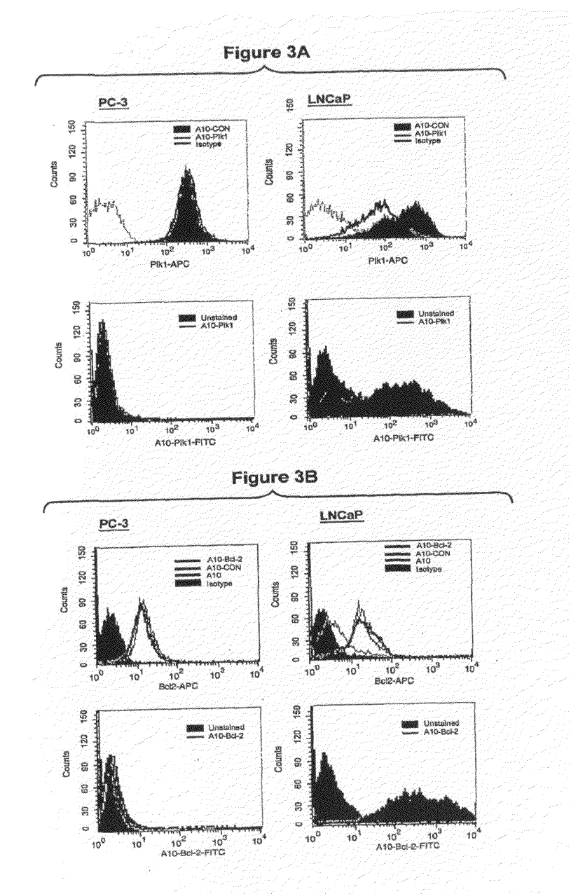

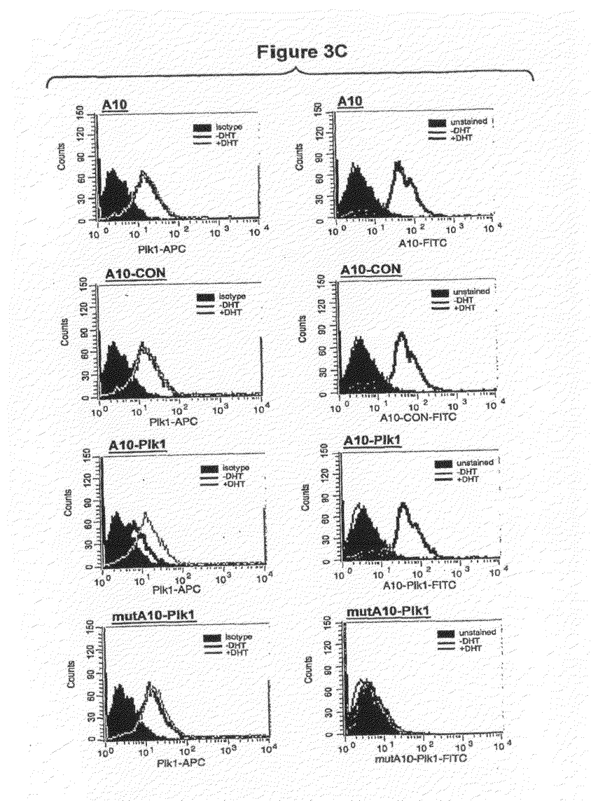

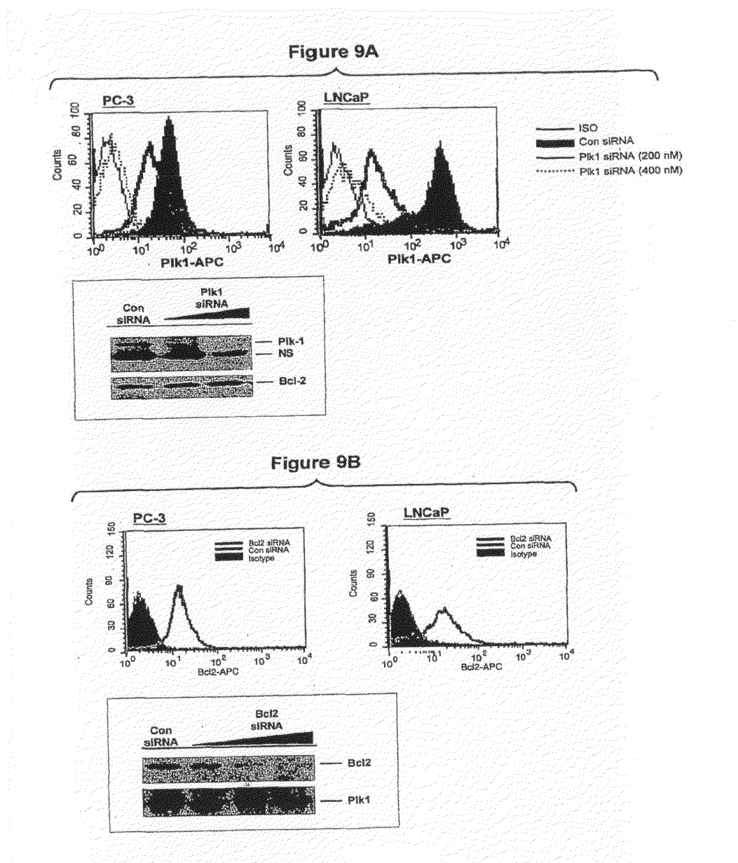

To determine whether the aptamer-siRNA chimeras can silence target gene expression, A10 aptamer-siRNA chimeras were used to deliver siRNAs against Plk1 (Reagan-Shaw and Ahmad, FASEB J. 19(6):611-3 (2005)) or Bcl2 Yano et al, Clin. Cancer Res. 10(22):7721-6 (2004)) to cells expressing PSMA (FIG. 3). PC-3 and LNCaP cells were treated with aptamer-siRNA chimeras A10-Plk1 (FIG. 3A), or A10-Bcl-2 (FIG. 3B) in the absence of transfection reagents. Silencing of Plk1 and Bcl-2 genes was assessed by flow cytometry and/or immunoblotting. In contrast to transfection of the non-targeted siRNAs (FIG. 9), silencing by A10-Plk1 and A10-Bcl-2 was specific to LNCaP cells expressing PSMA and correlated with uptake of fluorescent-labeled aptamer-siRNA chimeras into LNCaP cells (FIGS. 3A and 3B). The cell-type specific reduction in Plk1 and Bcl-2 proteins indicates that the siRNAs are being delivered specifically to PSMA expressing cells via the aptamer portion of the chimeras. To further verify that silencing by A10 aptamer-siRNA chimeras was indeed dependent on PSMA, LNCaP cells were incubated with or without 2 nM DHT for 48 h prior to addition of A10-Plk1 (FIG. 3C). Uptake of A10-Plk1 into cells and silencing of Plk1 gene expression were substantially decreased in cells treated with DHT. These data, together with the cell surface binding data, indicate that cell-type specific silencing is dependent upon cell surface expression of PSMA.

### Aptamer-siRNA Chimeras Inhibit Cell Proliferation and Induce Apoptosis of Cells Expressing PSMA.

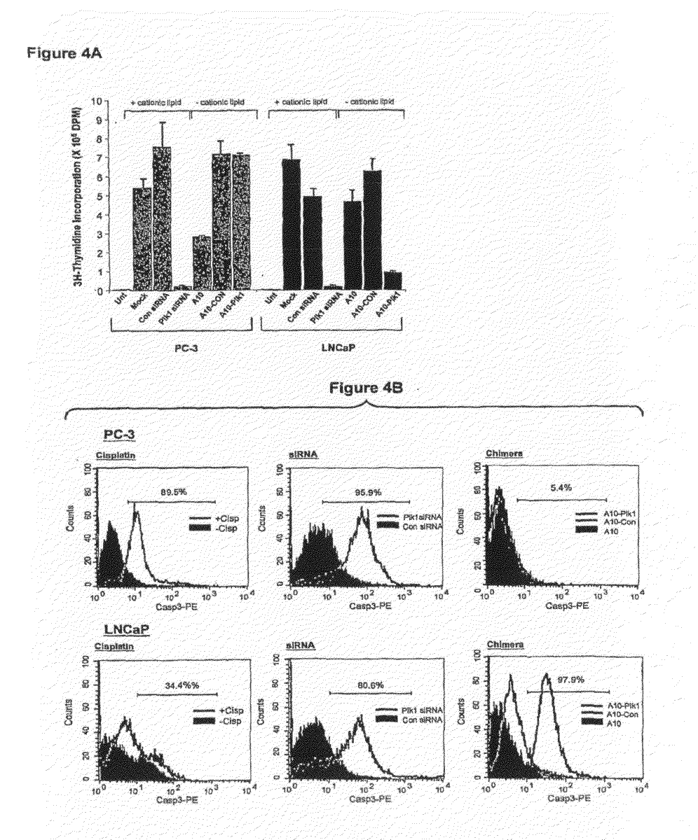

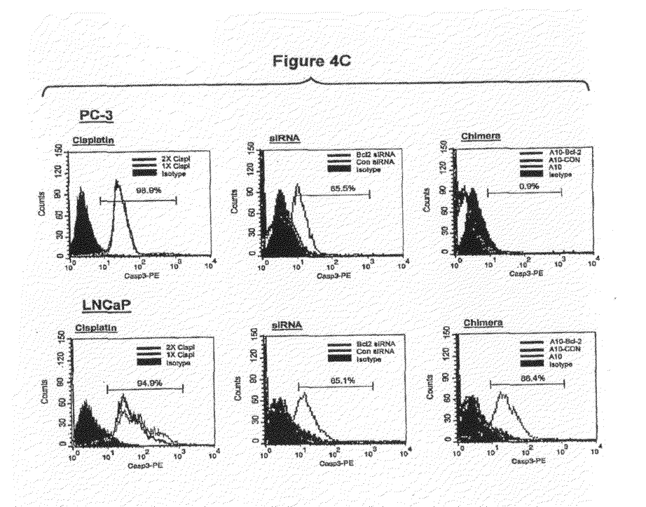

To determine whether the aptamer-siRNA chimeras targeting oncogenes and anti-apoptotic genes can achieve the goal of reducing cell proliferation and inducing apoptosis, these cellular processes were measured in cells treated with the chimeras. PC-3 and LNCaP cells were treated with A10-CON or A10-Plk1 aptamer-siRNA chimeras (FIG. 4A) and cell proliferation was measured by ³H-thymidine incorporation. In LNCaP cells, proliferation was effectively reduced by the A10-Plk1 chimera but not the control A10-CON chimera. This effect was specific for cells expressing PSMA as it was not seen in the PC-3 cells. Proliferation was reduced to nearly the same extent as observed when cationic lipids were employed to transfect Plk1 siRNA even though no transfection reagent was utilized for aptamer-siRNA delivery (FIG. 4A).

Next, the ability of the A10-Plk1 and A10-Bcl-2 chimeras to induce apoptosis of prostate cancer cells expressing PSMA was assessed (FIGS. 4B and 4C). PC-3 and LNCaP cells were either treated by addition of A10, A10-CON, A10-Plk1, or A10-Bcl2, to the media or transfected with siRNAs to Plk1 or Bcl2 using cationic lipids. Apoptosis was assessed by measuring production of active caspase 3 (Casp3) by Flow cytometry. While transfected siRNAs to Plk1 and Bcl-2 induced apoptosis of both PC-3 and LNCaP cells, apoptosis induced by the aptamer-siRNA chimeras was specific to LNCaP cells and did not require a transfection reagent. Treatment of PC-3 and LNCaP cells with cisplatin was used as a positive control for apoptosis.

### Aptamer-siRNA-Mediated Gene Silencing Occurs via the RNAi Pathway.

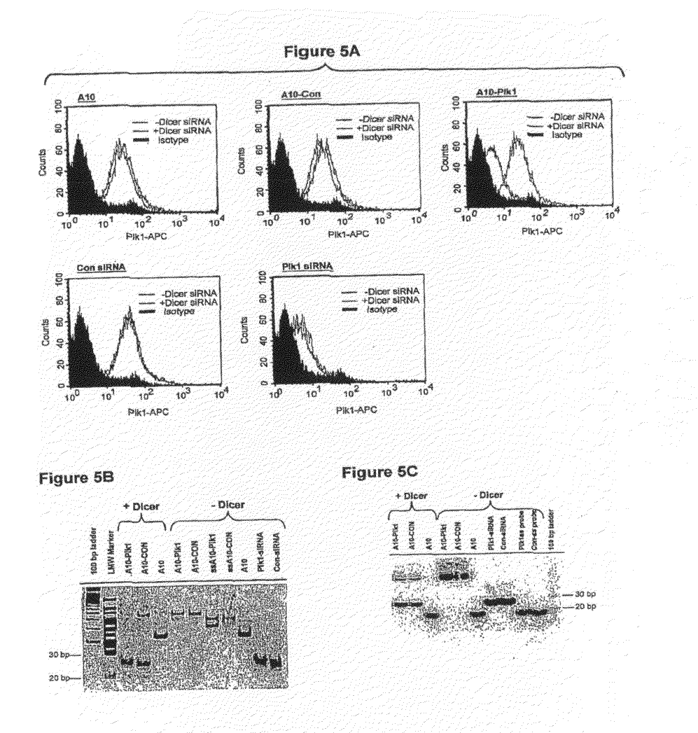

Next, a determination was made as to whether the mechanism by which aptamer-siRNA chimeras silence gene expression is dependent on Dicer activity. Therefore, the Dicer protein level was reduced by targeting its expression with an siRNA against human Dicer (Doi et al, Curr. Biol. 13(1):41-6 (2003)) (FIG. 10). Next, A10-Plk1 chimera-mediated gene silencing was tested for its dependence on Dicer expression. LNCaP cells were co-transfected with either A10 aptamer or aptamer-siRNA chimeras (A10-CON or A10-Plk1) alone or together with the Dicer siRNA (FIG. 5A). Silencing of Plk1 gene expression by the A10-Plk1 chimera was inhibited by co-transfection of Dicer siRNA (FIG. 5A, top panels) suggesting that aptamer-siRNA chimera-mediated gene silencing is dependent on Dicer and occurs via the RNAi pathway. In contrast, as expected, inhibition of Dicer had no effect on Plk1 siRNA-mediated silencing (FIG. 5A, bottom panels) because siRNAs of 21-23 nt in length have been shown to by-pass the Dicer step Murchison et al, Proc. Natl. Acad. Sci. USA 102(34):12135-40 (2005), Kim et al, Nat. Biotechnol. 23(2):222-6 (2005)).

To test whether the aptamer-siRNA chimeras were directly cleaved by Dicer to produce 21-23 nt siRNA fragments corresponding to the siRNA sequences engineered in the chimeric constructs the RNAs were digested with recombinant Dicer enzyme in vitro and the resulting fragments were resolved with non-denaturing PAGE (FIGS. 5B and 5C). As shown in FIG. 5B, the aptamer-siRNA chimeras (A10-CON or A10-Plk1), but not A10 or the longer single-stranded sense strand of the aptamer-siRNA chimeras (ssA10-CON or ssA10-Plk1), was digested by Dicer enzyme to release 21-23 nt fragments in length. To verify that these 21-23 nt long Dicer fragments correspond to the control and Plk1 siRNAs, the A10-aptamer-siRNA chimeras were labeled by annealing the complementary ³²P-end labeled anti-sense strand of the siRNAs and incubated with or without recombinant Dicer (FIG. 5C). Digest of labeled A10-CON or A10-Plk1 with recombinant Dicer resulted in release of 21-23 nt long fragments that retained the ³²P-end labeled anti-sense strand indicating that these fragments are indeed the siRNA portion of the aptamer-siRNA chimeras.

### Aptamer-siRNA Chimeras do not Trigger Interferon Responses.

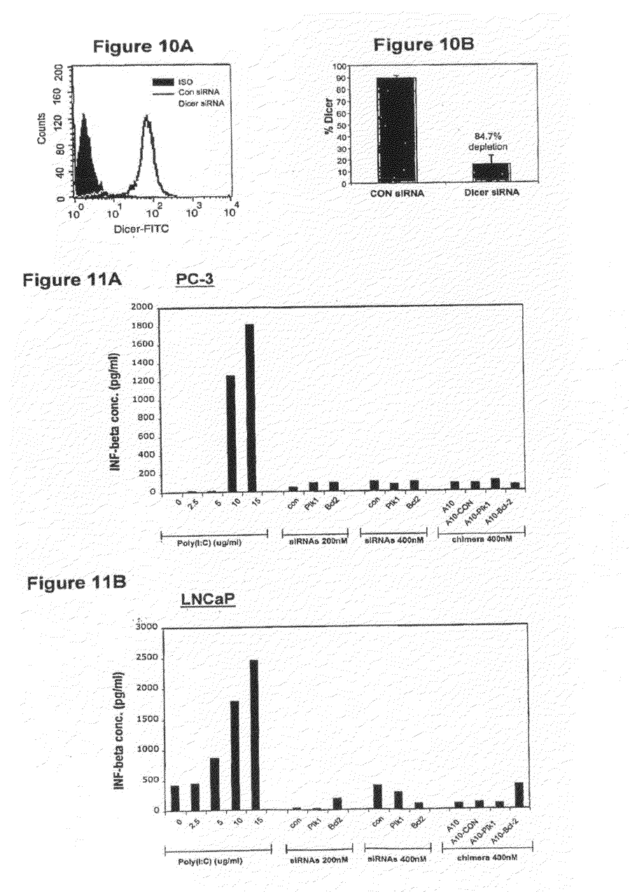

Various groups have reported that delivered siRNAs can potentially activate non-specific inflammatory responses, leading to cellular toxicity (Sledz et al, Nat. Cell Biol. 5(9):834-9 (2003), Kariko et al, J. Immunol. 172(11):6545-9 (2004)). Therefore, a determination was made of the amount of INF-β produced by PC-3 and LNCaP cells that were either untreated, transfected with siRNAs to Plk1 or Bcl-2, or treated with the aptamer-siRNA chimeras using an enzyme-linked immunosorbant assay (ELISA) (FIG. 11). Treatment with either siRNAs or aptamer-siRNA chimeras did not induce production of INF-β under these experimental conditions suggesting that delivery of aptamer-siRNA chimeras to cells does not trigger a substantial interferon response.

### A10-Plk1 Mediates Tumor Regression in a Mouse Model of Prostate Cancer.

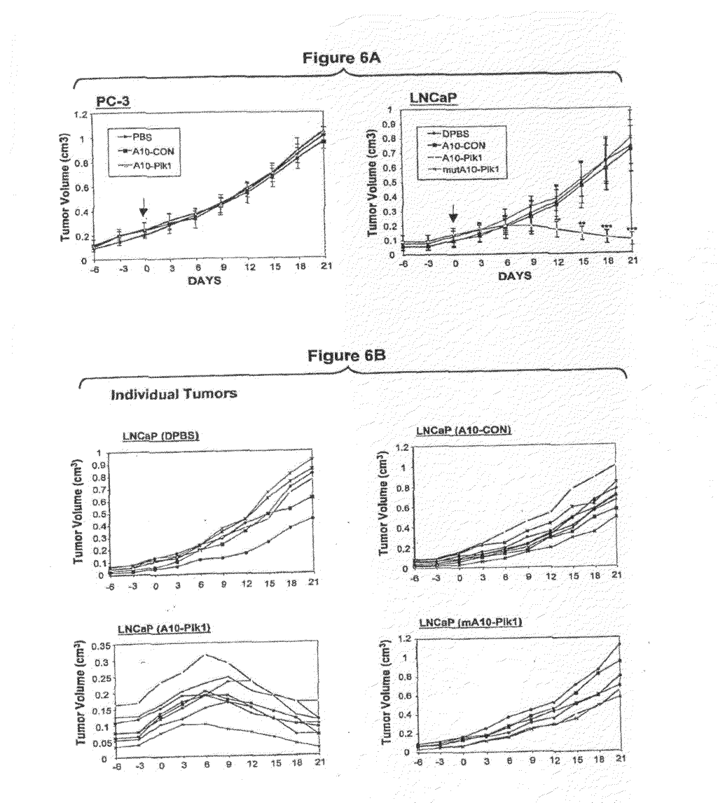

The efficiency and specificity of the A10-Plk1 chimera in athymic mice bearing tumors derived from either PSMA positive human prostate cancer cells (LNCaP) or PSMA negative human prostate cancer cells (PC-3) was addressed next (FIG. 6). Athymic mice were inoculated with either LNCaP or PC-3 cells and tumors were allowed to grow until they reached 1 cm in diameter in the longest dimension. Tumors were then injected (Day 0) with either DPBS alone or with the chimeric RNAs (A10-CON, A10-Plk1, or mutA10-Plk1) every other day for a total of ten injections administered. Tumors were measured every three days. No difference in tumor volume was observed with the PC-3 tumors with any of the different treatments indicating that the chimeric RNAs did not have any non-specific cell killing effect. In contrast, a pronounced reduction in tumor volume was observed for LNCaP tumors treated with A10-Plk1 chimera. Indeed, from Day 6 to Day 21 the various control treated tumors increased 3.63 Fold in volume (n=22) while the A10-Plk1 treated had a 2.21 Fold reduction in volume (n=8). Regression of LNCaP tumor volume was specific to the A10-Plk1 and was not observed with DPBS treatment or treatment with the A10-CON or mutA10-Plk1 chimeric RNAs. Importantly, no morbidity or mortality was observed following the 20-day treatment with the chimeric RNAs suggesting that these compounds are not toxic to the animals under the conditions of these experiments.

In summary, aptamer-siRNA chimeras have been developed and characterized that target specific cell types and act as substrates for Dicer thereby triggering cell-type specific gene silencing. In the above-described study, anti-apoptotic genes were targeted with RNAi specifically in cancer cells expressing the cell-surface receptor, PSMA. Depletion of the targeted gene products resulted in decreased proliferation and increased apoptosis of the targeted cells in culture (FIG. 4). Cellular targeting of the chimeric RNAs was mediated by the interaction of the aptamer portion of the chimeras with PSMA on the cell surface. Significantly, a mutant chimeric RNA bearing two point mutations within the region of the aptamer responsible for binding to PSMA resulted in loss of binding activity (FIG. 1D). Binding specificity was further verified by demonstrating that PC-3 cells, which do not express PSMA, and LNCaP cells depleted of PSMA by treatment with 5-α-dihydrotestosterone were not targeted by the chimeras, whereas untreated LNCaP cells, which express PSMA, were targeted (FIG. 2C). Additionally, antibodies specific for PSMA competed for binding of the chimeras to the LNCaP cell surface (FIG. 2B).

It has been shown that gene silencing by the chimeric RNAs is dependent on the RNAi pathway because it requires Dicer, an endonuclease that processes dsRNAs prior to assembly of RISC complexes (FIG. 5A). Dicer was also found to cleave the double-stranded, gene-targeting portion of the chimeras from the aptamer portion, a step that would be expected to precede incorporation of the shorter strand of these reagents into RISC complexes (FIGS. 5B and 5C).

Importantly, this siRNA delivery approach effectively mediated tumor regression in a mouse model of prostate cancer (FIG. 6). The RNA chimeras are therefore suitable for targeting tumors in mice in vivo in the form in which they have been generated and may, in the future, prove to be useful therapeutics for human prostate cancer. These reagents exhibited the same specificity for PSMA expression in vivo as they did in vitro as the PSMA-negative PC-3 tumors did not regress when treated. It is noteworthy that the RNA used to make the chimeras is protected from rapid degradation by extracellular RNAses by the 2′-fluoro modification of the pyrimidines in the aptamer sense strand, which is likely to be essential for their performance in vivo (as well as in vitro in the presence of serum) (Allerson et al, J. Med. Chem. 48(4):901-4 (2005), Layzer et al, RNA 10(5):766-71 (2004), Cui et al, J. Membr. Biol. 202(3):137-49 (2004)).

While various methods have been described for delivering siRNAs to cells, most of these methods accomplish delivery non-specifically (Yano et al, Clin Cancer Res. 10(22):7721-6 (2004), Fountaine et al, Curr Gene Ther. 5(4):399-410 (2005), Devroe and Silver, Expert Opin Biol Ther. 4(3):319-27 (2004), Anderson et al, AIDS Res Hum Retroviruses. 19(8):699-706 (2003), Lewis and Wolff, Methods Enzymol. 392:336-50 (2005), Schiffelers et. al. Nucleic Acids Res. 32(19):e149 (2004), Urban-Klein et al, Gene Ther. 12(5):461-6 (2005), Soutschek et al, Nature 432(7014):173-8 (2004), Lorenz et al, Bioorg Med Chem Lett. 14(19):4975-7 (2004), Minakuchi et al, Nucleic Acids Res. 32(13):e109 (2004), Takeshita et al, Proc Natl Acad Sci USA. 102(34):12177-82 (2005)). Cell-type specific delivery of siRNAs is therefore, a critical goal for the widespread applicability of this technology in therapeutics due to both safety and cost considerations.

All documents and other information sources cited above are hereby incorporated in their entirety by reference.

What is claimed is:

1. A chimeric molecule comprising a nucleic acid targeting moiety and an RNA silencing moiety, wherein said molecule is a Dicer substrate.

2. The molecule according to claim 1 wherein said targeting moiety is an aptamer.

3. The molecule according to claim 1 wherein said targeting moiety targets a cell surface receptor.

4. The molecule according to claim 1 wherein such targeting moiety targets PSMA, Plk1 or Bcl2.

5. The molecule according to claim 1 wherein said molecule is an RNA molecule.

6. The molecule according to claim 1 wherein said molecule comprises an aptamer and a pre-siRNA, an aptamer and a shRNA, an aptamer and a pre-miRNA or an aptamer and a pri-miRNA.

7. A composition comprising the molecule according to claim 1 and a carrier.

8. A method of effecting targeted delivery to a cell of an RNA silencing moiety comprising contacting a cell comprising a target recognized by a targeting moiety with the chimeric molecule according to claim 1 under conditions such that said cell internalizes said molecule and Dicer present in said cell processes said molecule so that said silencing is thereby effected.

9. The method according to claim 8 wherein said cell is a cell in vivo.

10. The method according to claim 9 wherein said cell is a human cell.

11. The method according to claim 10 wherein said cell is a cancer cell.

12. The method according to claim 11 wherein said cell is a prostate cancer cell.
